(19) United States
(12) Patent Application Publication
(10) Pub. No.: US 2010/0298393 A1
(43) Pub. Date: Nov. 25, 2010

(54) Modulation of Growth Hormone, DHEA, and Cortisol with Positive Modulators of AMPA Type Glutamate Receptors

(76) Inventors: Peter Vanderklish, San Diego, CA (US); Keith Hoffman, San Diego, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/678,477

(22) PCT Filed: Sep. 17, 2008
(86) PCT No.: PCT/US08/76618

§ 371 Date: Aug. 10, 2010

Related U.S. Application Data
(60) Provisional application No. 60/973,089, filed on Sep. 17, 2007.

(51) Int. Cl. A61K 31/42; A61P 5/00

(52) U.S. Cl. 514/380

(57) ABSTRACT

Methods for modulating the endocrine system of a mammal are provided. In the subject methods, a positive allosteric modulator of AMPA receptors of the hypothalamus are administered to the host. The subject methods find use in applications where it is desired to increase the baseline circulatory level of a growth hormone in a mammalian host. The subject methods also find use in applications where it is desired to increase the circulatory level of DHEA in a mammalian host. Finally, another subject method also finds use in applications where it is desired to decrease the circulatory level of cortisol in a mammalian host.

### CROSS-REFERENCE To RELATED APPLICATIONS

Pursuant to 35 U.S.C. §119 (e), this application claims priority to U.S. Provisional Application Ser. No. 60/973,089 filed Sep. 17, 2007; the disclosure of which priority application is herein incorporated by reference.

### INTRODUCTION

The endocrine system regulates all aspects of somatic function during development and maturity, including growth, sexual maturation, metabolism, immune function and stress responses. Endocrine function also impacts the central and peripheral nervous systems through feedback and via local release of hormones and releasing factors within nervous tissues. With ageing, the synthesis and activity of several hormone systems becomes progressively dysregulated, resulting in abnormalities in both the amplitude and timing of hormone pulses that occur in a cyclic manner throughout the day. Prominent examples of this are age-related declines in the levels of GH, and exaggerated cortisol spikes in response to stressors. The consequences of this decline in endocrine function range from decreased muscle mass to impaired cardiovascular, reproductive, immune and cognitive function.

The reasons for endocrine regulatory decline are multifold, but are likely to include mechanisms of neuronal compromise evident in many brain regions during the aging process. Direct pharmacological stimulation of AMPA receptors can trigger the release of several hormones, including GH, and antagonism of AMPA and NMDA receptors impairs endocrine function. This raises the possibility that age-related pathology of glutamatergic synapses is in part responsible for the compromise in endocrine function. We were the first to propose the idea of using positive allosteric modulators of AMPA receptors as a means of enhancing endocrine function, and addressed this idea in experiments supporting U.S. Pat. No. 6,329,368 and No. 6,620,808 “Endocrine modulation with positive modulators of AMPA type glutamate receptors”.

Positive allosteric modulators of AMPA-type glutamate receptors have been developed as means of enhancing glutamatergic transmission in brain. By reducing desensitization and deactivation of AMPA receptors, these drugs enhance fast excitatory currents at single synapses, the fidelity of transmission through polysynaptic glutamatergic circuits, and the production of activity-dependent trophic factors such as BDNF that enhance neuronal viability and synaptic communication over longer time periods. We demonstrated previously that single doses of members of the Ampakine class of positive allosteric modulators elevated serum GH levels in rats. In parallel in vitro studies, we found that incubation of cultured hypothalamic slices with the same drug enhances the secretion of GHRH into the culture medium.

### SUMMARY

Methods for modulating mammalian endocrine systems are provided. In the subject methods, allosteric modulators of AMPA receptors of the hypothalamus, e.g. agents belonging to the “ampakine” family of compounds, are administered to a subject. The subject methods find use in a variety of different applications where modulation of the endocrine system of a mammal is desired, such as in the treatment of diseases associated with hormonal system dysfunction, particularly with abnormally decreased baseline levels of a hormone, e.g., growth hormone, or DHEA, etc. or with abnormally increased levels of a hormone, e.g., stress hormones such as corticosterone, cortisol, etc.

Aspects of the invention include treating a host for hormonal imbalances and therapeutic regimes for treating low levels of baseline growth hormone in a host, including therapeutic regimes for treating low levels of DHEA in a host. Additional aspects of the invention include therapeutic regimes for treating high levels of a stress hormone, such as cortisol, in a host. In certain embodiments, a host's endogenous hormones are those that are modulated. Embodiments of the invention do not involve the oftentimes-dangerous technique of exogenous hormone administration. In certain embodiments, formulations with specific positive modulators of AMPA type glutamate receptors are employed.

In the studies presented below, we tested the effects of repeated dosing with members of two distinct families of AMPA receptor modulators on GH levels in rats—on two new parameters—baseline circulatory and peak concentrations. The results show that both families of AMPA receptor modulators increased the morning GH pulse and baseline levels of GH. Significant elevations in GH levels were observed in young adult and middle-aged rats. The results indicate that the GHRH-GH-IGF1 axis can be enhanced by positive modulation of AMPA receptors, and that this strategy will be useful in offsetting age-related declines in the function of this system and other hormone cascades regulated centrally by glutamatergic transmission in a variety of different organisms, such as mammalian organisms.

To extend the characterization of the effects that facilitation of AMPA receptors has on hormone secretion, we also analyzed the effects of positive modulation of the AMPA receptor on levels of the adrenal androgen dihydroepiandrosterone (DHEA) and the stress hormone corticosterone. DHEA and corticosterone (the rat equivalent of cortisol in humans) were investigated because of their relationship to ageing and a wide range of somatic and central nervous system disorders. During ageing, levels of DHEA decline gradually to approximately 20% of post-pubescent levels by the age of 70. Levels of the stress hormone cortisol, in contrast, do not decline; instead, they exhibit an age related increase in nocturnal baseline levels and in the size of peak secretion events linked to psychosocial stress. The net outcome of these changes is an elevated ratio of cortisol to DHEA levels, which favors a catabolic state and many believe may both trigger and promote age-related declines. This idea is supported by i) the known functions of DHEA and cortisol, ii) the fact that the onset of changes in DHEA and cortisol precede several measures of age-related decline, and iii) strong correlations between DHEA levels and successful ageing.

The adverse consequences of an elevated cortisol to DHEA ratio, and the potential benefits of normalizing this, are numerous and diverse. Illustrative of this is the fact that elevations in this ratio have been correlated with the severity of age-related declines in cognitive function, cell and antibody-mediated immunity, muscle mass, cardiovascular health and other systems. Such changes are related to both the primary actions of cortisol and DHEA, and to secondary and tertiary effects of these hormones. For instance, DHEA can exert many of its functions via conversion to sex steroids, and both cortisol and DHEA have been shown to regulate the growth hormone (GH)—insulin-like growth factor 1 (IGF1) axis. Elevated cortisol and reduced DHEA levels impair the function of these peripheral hormone systems and, in particular, reduce the effectiveness of GH. DHEA is also known to have anti-cortisol effects in target tissues, which suggests that reductions in DHEA levels can potentiate the deleterious effects of abnormally high cortisol. In addition, it is well established that elevated cortisol levels predispose neurons in the hippocampus and other regions of telencephalon to degeneration in response to a number of acute and chronic conditions. DHEA, conversely, has been shown to promote neuronal viability in these areas.

The deleterious effects of an elevated cortisol-DHEA ratio on hippocampal function are of particular significance because of the role the hippocampus plays in learning and memory, and its regulatory influence on the entire hypothalamic-pituitary axis.

It is well established that a number of hormone systems governing body mass, reproduction, stress responses, immunity and basic metabolism become progressively dysregulated during the ageing process. Glutamatergic transmission in the hypothalamus, arising from both intra- and extrahypothalamic efferents, regulates hormone levels by controlling the secretion of releasing factors. We demonstrate here that a progressive deterioration of glutamatergic transmission in the hypothalamus contributes to endocrine dysregulation, and that positive allosteric modulators of AMPA-type glutamate receptors are efficacious in reversing such deficits. Previously, we demonstrated in rats that single injections of a member of the Ampakine class of AMPA receptor positive modulators increase serum growth hormone (GH) levels, and that incubation of cultured hypothalamic explants with an Ampakine increases the release of growth hormone releasing hormone (GHRH). In the present study, we assayed the effects of longer-term treatment of rats with AMPA receptor positive modulators on serum growth hormone levels—looking for the first time at baseline and peak morning pulses of growth hormone. We also assayed the effect of AMPA receptor positive modulators on the levels of DHEA and Cortisol in treated subjects.

Two distinct classes of positive modulators increased serum GH levels in both young adult rats injected with the drugs daily over a four day period, and in middle aged rats treated daily for twelve days. Increases were observed in morning growth hormone pulses occurring immediately after drug injection, and in baseline levels before drug injection. Further, the two distinct classes of AMPA positive modulators had significant effects on the levels of both DHEA and Cortisol in treated subjects.

The results show that positive modulation of AMPA receptors is a viable strategy for upregulating baseline levels and peak concentrations of growth hormone secretion. The results further show that positive modulation of AMPA receptors is a viable strategy for upregulating DHEA levels and for decreasing levels of circulating stress hormones, such as cortisol.

### BRIEF DESCRIPTION OF THE FIGURES

FIG. 1 is a graph depicting the effects of positive AMPA receptor modulators on GH levels in the morning GH peak in young adult rats.

FIG. 2 is a graph depicting the effects of positive AMPA receptor modulators on morning baseline GH levels in middle-aged rats.

FIG. 3 is a graph depicting the effects of positive allosteric modulators of AMPA receptors on blood DHEA levels.

FIG. 4 is a graph depicting the effects of positive allosteric modulation of AMPA receptors on blood corticosterone levels in middle aged rats.

### DETAILED DESCRIPTION

Methods of modulating the endocrine system of a mammalian host are provided. In the subject methods, a therapeutically effective amount of an agent or positive modulator which enhances the effect of excitatory amino acid transmitters on the AMPA type glutamate receptor and which crosses the blood-brain barrier, e.g. a compound belonging to the ampakine family of compounds, is administered to a mammalian host. The subject methods find use in a variety of applications where regulation of the mammalian endocrine system is desired, such as in the treatment of diseases associated with either low, or high, circulatory levels of a given endocrine-related hormone.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In the subject methods, a blood brain permeable positive modulator of AMPA receptors, e.g., an ampakine, is administered to a mammalian host to modulate the levels of a target hormone in a mammalian host. Compounds suitable for use in the subject methods are generally those that amplify (up-modulate) the activity of AMPA receptors. Compounds suitable for such use may be identified by the assay systems described in U.S. Pat. No. 6,620,808, and are hereby incorporated by reference.

The central action of a compound can be verified by measurement of synaptic responses or the overall activity of brain cells in behaving animals (see Staubli et al., 1994a) and time course of biodistribution can be ascertained via injection and subsequent quantitation of drug levels in various tissue samples. Quantitation can be accomplished by methods known to those skilled in the art and will vary depending on the chemical nature of the drug.

Compounds useful in the practice of this invention are generally those that amplify the activity of the natural stimulators of AMPA receptors, particularly by amplifying excitatory synaptic response as defined above. They are quite varied in structure and so long as they embrace the above physiological properties and cross the blood brain barrier, they will work in this invention. Preferred compounds include, but are not limited to, compounds identifiable by the assays described above.

Specific compounds of interest for use in embodiments of the invention (and methods of how to make them) include, but are not limited to, those compounds published in U.S. Pat. No. 6,620,808, the disclosure of which compounds is herein incorporated by reference. Also of interest are the ampakine compounds disclosed in: Trends Neurosci. 2006 October;29(10):554-62; Curr Opin Pharmacol. 2004 February;4(1):4-11; Drugs. 2000 January;59(1):33-78; Psychopharmacology (Berl). 2005 April;179(1):4-29; Curr Opin Pharmacol. 2006 February;6(1):82-8; Curr Drug Targets CNS Neurol Disord. 2004 June;3(3):181-94; CNS Drug Rev. 2002 Fall;8(3):255-82; Neuropharmacology. 2001 June;40(8):976-83; Neuropharmacology. 2001 June;40(8):1003-9; Curr Drug Targets. 2007 May;8(5):603-20; Bioorg Med Chem Lett. 2006 Oct. 1;16(19):5057-61; Curr Drug Targets CNS Neurol Disord. 2004 June;3(3):181-94. All of the compounds disclosed in these publications are incorporated herein by reference.

Turning now to the subject methods, in the subject methods, a therapeutically effective amount of one or more, usually no more than 5, more usually no more than 2, types of distinct ampakine compounds, as described above, are administered to a mammalian host in which modulation of the endocrine system is desired. The term “therapeutically effective amount” means an amount effective to cause a modulation or alteration in the endocrine system of the host being treated, usually by changing the blood levels of one or more particular hormones.

The compounds of this invention can be incorporated into a variety of formulations for therapeutic administration, i.e. combined with a physiological acceptable vehicle to produce a pharmaceutical composition. Examples of suitable pharmaceutical compositions include capsules, tablets, syrups, suppositories, and various injectable forms. Administration of the compounds can be achieved in various ways, including oral, bucal, rectal, parenteral, intraperitoneal, intradermal, transdermal administration. Formulations of interest of the compounds are oral preparations, particularly capsules or tablets.

Dose levels can vary as a function of the specific compound, the severity of the symptoms, and the susceptibility of the subject to side effects. Some of the specific compounds that stimulate glutamatergic receptors are more potent than others. Preferred dosages for a given compound are readily determinable by those of skill in the art by a variety of means. A preferred means is to measure the physiological potency of a given compound that is a candidate for administration, and such methods are discussed in U.S. Pat. No. 6,620,808, and are hereby incorporated by reference.

The above described compounds and/or compositions are administered at a dosage sufficient to achieve the desired modulation of endocrine system while minimizing any side-effects. Typical dosages for systemic administration range from 0.1 to 10 milligrams per kg weight of subject per administration.

The dosing may be scheduled as desired, where a dose is administered periodically over a given period of time, e.g. once a day for a month or longer, twice a day on a daily basis for two weeks, and the like. For example, a typical dosage may be one 10-50 mg tablet taken once a day, or one time-release capsule or table taken once a day and containing a proportionally higher content of active ingredient. The time-release effect may be obtained by capsule materials that dissolve at different pH values, by capsules that release slowly by osmotic pressure, or by any other known means of controlled release.

In certain embodiments, the methods of the invention are methods for increasing the baseline circulatory levels of growth hormone in a mammalian host. By baseline level is meant that levels of growth hormone occurring at any time of day between circadian linked peaks in growth hormone. The magnitude of increase of the baseline level that is achieved in embodiments of the invention is 1.25 fold or more, such as two fold or more and including four fold or more. Accordingly, embodiments of the invention may be employed to achieve baseline circulatory levels of growth hormone in an equivalent of a 60 year hold human that range from 1 μg/L to 2 μg/L, such as 0.75 μg/L to 1 μg/L and including 2 μg/L to 4 μg/L.

In certain embodiments, the methods of the invention are methods for increasing the peak level of growth hormone in a mammalian host. By peak level is meant the levels of growth hormone accompanying circadian-linked increases in growth hormone release induced by endogenous factors such as growth hormone releasing hormone. The magnitude of increase of the peak level that is achieved in embodiments of the invention is 1.25 fold or more, such as 1.5 fold or more and including two fold or more. Accordingly, embodiments of the invention may be employed to achieve peak levels of growth hormone in an equivalent of a 60 year hold human that range from 2.5 μg/L to 12 μg/L, such as 3 μg/L to 8 μg/L and including 8 μg/L to 12 μg/L.

In certain embodiments, the methods of the invention are methods for increasing the circulatory level of DHEA in a mammalian host. By circulatory level is meant the amount of DHEA in the blood stream. The magnitude of increase of the circulatory level that is achieved in embodiments of the invention is 1.5 fold or more, such as two fold or more and including three fold or more. Accordingly, embodiments of the invention may be employed to achieve circulatory levels of DHEA in an equivalent of a 60 year hold human that range from 1.5 μg/L to 4 μg/L such as 2 μg/L to 3 μg/L and including 3.5 μg/L to 4 mg/mL.

In certain embodiments, the methods of the invention are methods for decreasing the circulatory level of cortisol in a mammalian host. By circulatory level is meant the amount of cortisol in the blood stream. The magnitude of decrease of the circulatory level that is achieved in embodiments of the invention is 0.2 fold or more, such as 0.25 fold or more and including 0.50 fold or more. Accordingly, embodiments of the invention may be employed to achieve circulatory levels of cortisol in an equivalent of a 60 year hold human that range from 400 mmol/L to 350 mmol/L or lees in the morning hours to 200 mmol/L to 50 mmol/L or less in mid day to night, such as 300 mmol/L in the early morning and 50 mmol/L at night.

The subject methods may be used to modulate the activity of the endocrine system, or subset or portion thereof, of a variety of different mammalian hosts. Mammalian hosts which may be treated according to the subject methods include rare and/or valuable animals, domestic animals, such as livestock, e.g. horses, cows, pigs, sheep, and the like; and pets, e.g. dogs, cats, and the like; and humans.

The term endocrine system as used in this application refers in general to the hormonal cell-cell communication system of the mammal. By modulation of the endocrine system is meant that the hormonal cell-cell communication of the mammal is altered in some manner, usually through a modulation or change in the blood circulatory level of one or more endogenous hormones, where modulation includes both increasing and decreasing the circulatory level of one or more hormones, usually increasing the circulatory level of one or more hormones, in response to the administration of the ampakine compound. Usually the subject methods are employed to modulate the activity of a particular hormonal system of the endocrine system of the mammal, where hormonal systems of interest include those which comprise glutamatergic regulation, particularly AMPA receptor regulation, where the hypothalamus-pituitary hormonal system is of particular interest.

Within the hypothalamus-pituitary system, of interest is the modulation of the following hormones: GHRH, GH, DHEA, cortisol, corticosterone, and the like.

The subject methods find use in a variety of diverse applications where one wishes to modulate a mammalian endocrine system. Representative applications in which the subject methods find use include the treatment of diseases associated with or resulting from the dysfunction of the endocrine system, where dysfunction refers to hyper or hyposecretion of one or more specific hormones, usually hyposecretion of one or more hormones. The terms “treatment,” “treating,” and “treat” and the like are used herein to generally mean obtaining a desired pharmacology and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” therefore includes: (a) preventing the disease or symptom from occurring in a subject which may be predisposed to the disease or symptom but has not yet been diagnosed as having the disease or symptom; (b) inhibiting the disease symptom, i.e. arresting its development; or (c) relieving the disease symptom, i.e. causing regression of the disease or symptom.

Of particular interest is use of the subject methods to treat diseases associated with dysfunction of the hypothalamus-pituitary hormonal system, where the dysfunction of this particular system results in the hyposecretion of one or more pituitary hormones, where the pituitary hormones are usually under the regulatory control of a neuropeptide secreted by the hypothalamus, particularly a neuropeptide secreted in response to binding of glutamate to an AMPA receptor of the hypothalamus.

Of particular interest is use of the subject methods to upregulate the production of endogenous hormone by the pituitary, where disease has resulted in a down regulation of hormone production by down regulating the production of the requisite hypothalamic stimulatory hormone. Thus, in this class of diseases, by administering ampakine comprising pharmaceutical compositions to the host, one upregulates the production of the stimulatory hypothalamic neuropeptide, which in turn upregulates the production of endogenous hormone by the pituitary, thereby increasing the circulatory levels of the hormone in the host.

Accordingly, one class of diseases which may be treated according to the subject methods are diseases associated with hyposecretion of growth hormone, resulting in abnormally low circulatory levels of growth hormone in the mammal, where the hyposecretion is not the result of substantially complete failure in the capability of the pituitary to produce growth hormone. By treating is meant that the subject methods result in an elevated circulatory level of growth hormone compared to the level prior to treatment.

One “disease” characterized by such down regulation of endogenous growth hormone production is aging. Therefore, the subject methods find use in the treatment of symptoms associated with aging, such as reduction in lean body mass, bone, muscle and the like. Other diseases associated with depressed blood circulatory growth hormone levels which may be treated by the subject methods include hyposomatotropism resulting from tumors, trauma, infections, and the like.

In certain embodiments, the methods include a step of diagnosing the need for treatment according to the methods in a patient. As such, the methods include a step of determining that a patient is in need of treatment according to the invention. In certain embodiments, the methods are performed on patients that have been diagnosed or determined to be in need of the treatment. As such, the methods include a step of providing a patient that has been diagnosed to be in need of the treatment.

The following examples are offered by way of illustration and not by way of limitation.

### EXPERIMENTAL

### A. Methods

Animal Treatments and Blood Sampling

Adult male Sprague Dawley rats with jugular vein canulas were purchased from Charles River; canulas were implanted by the vendor. Rats were acclimated for a 3-5 day period with regular handling prior to the studies. The effects of positive modulators of AMPA-type glutamate receptors on serum GH levels were evaluated in two separate studies, each using compounds developed by Cortex Pharmaceuticals (CX1) and Eli Lilly (LY1 and LY2). In the first study (Study 1), 24 young adult rats (3 months old with an average weight of 315 g) were divided equally into 4 treatment groups, those receiving: vehicle (16.5% β-cyclodextrin), CX1 (33 mg/kg), LY1 (0.5 mg/kg), or LY2 (1 mg/kg). On each of 4 consecutive days, rats were treated with vehicle or positive modulators by i.p. injection between 10:00 and 10:30 AM, and 300 ul samples of blood were collected via the jugular vein canulas at 0, 30, 60 and 90 minutes post injection. The samples were spun for 5 minutes at 2000×g to obtain cleared serum, which was then stored at −80° C. until they were analyzed for GH levels by ELISA. In the second study (Study 2), 12 middle-aged rats (8-9 months old with an average weight of 390 grams) were divided equally into 3 groups, those receiving: vehicle, CX1 (33 mg/kg), or LY1 (0.5 mg/kg). On each of 12 consecutive days, rats were injected i.p. with vehicle or positive modulators between 8:30 and 8:50 AM. On days 1, 2, 4, 5, 8, 9, 11, and 12, blood samples were collected prior to injection and at 30, 60, and 90 minutes post injection. To optimize the timing of blood collection, sampling was staggered such that blood was collected from 2 animals in each group on a given day. Serum samples were stored at −80° C. until analysis.

Analysis of serum GH levels by ELISA: Competitive ELISA kits (96 well format) for the analysis of rat GH were obtained from ALPCO Diagnostics (Salem, N.H.). The assays had a detection limit of ˜0.35 ng/ml and inter- and intra-assay coefficients of variation of 4% and 15%, respectively. Frozen samples were thawed overnight at 4° C. Each assay plate was set up to include all the timepoints (4 samples) of a given animal in each treatment group (4 groups in study 1 and 3 in study 2), an 8 point standard curve (0.35 ng/ml to 40 ng/ml rat GH), quality control samples, and blanks. For each set of 4 timepoints samples were run in duplicate without dilution, and also in duplicate with a 1:11 dilution in assay buffer—to ensure that GH readings would be captured within the range of the standard curve. Care was taken to minimize variation due to volume transfer by using a calibrated multichannel pipette. After setting up and processing the ELISA plates according to the manufacturer's recommendations, the plates were read at 410 nm with correction at 910 nm; readings were taken after 90 minutes of incubation with development substrate when the OD of the maximal binding wells was approximately 0.5 units.

Data Analysis:

All data were assembled, analyzed and graphed using Microsoft Excel and Graphpad Prism. GH concentrations interpolated from the standard curve were plotted as a function of sampling time. In Study 1, timecourses that captured defined peaks were selected for analysis of group data. Average area under the curve measurements in CX1, LY1 and LY2 groups were compared to the vehicle control group and analyzed for statistical significance. In Study 2, analysis focused on the early AM baseline levels of GH as the majority of timecourses did not capture an entire defined peak. Baselines of drug treatment groups were expressed as a percentage of control values on a given sampling day, and these values were averaged across the length of the study.

The methods employed to assay levels of corticosterone and DHEA are the same as those used to measure GH and, thus, will not be described in detail. It is important to note, however, that the samples from Study 1 (young adult rats) and Study 2 (middle aged rats) that we analyze here are the same samples used to measure GH levels. Thus, we have determinations of the levels of three hormones from the same animals, at the same times, and under the same conditions. DHEA levels were determined in samples from both studies, but due to an insufficient sample volume cortisol levels were measured only in samples from Study 2.

### B. Results—Growth Hormone

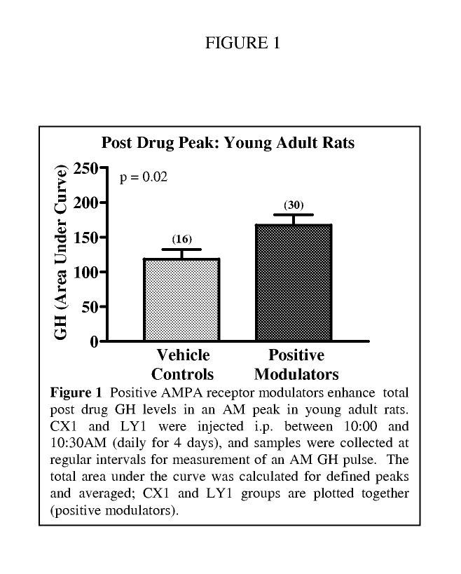

Study 1 The effects of AMPA receptor modulators were evaluated in two studies (see Methods). In the first, 24 young adult rats were divided equally among four treatment groups, those receiving: vehicle, CX1, LY1, and LY2. Rats were dosed every day for 4 days. GH levels in samples collected at 0, 30, 60 and 90 minutes after drug injection were assayed by ELISA and plotted as a function of time. Defined peaks were then analyzed for total area under the curve and peak GH levels. Significant increases in the level of serum GH relative to vehicle were obtained using CX1 and LY1, but not LY2. The effects of both CX1 and LY1 were comparable and are represented together in FIG. 1. The average area under the GH curve values for CX1 and LY1 combined was increased by approximately 45% relative to vehicle controls (p=0.02). LY1, but not CX1, also induced significant increases in peak GH (p<0.05; not shown).

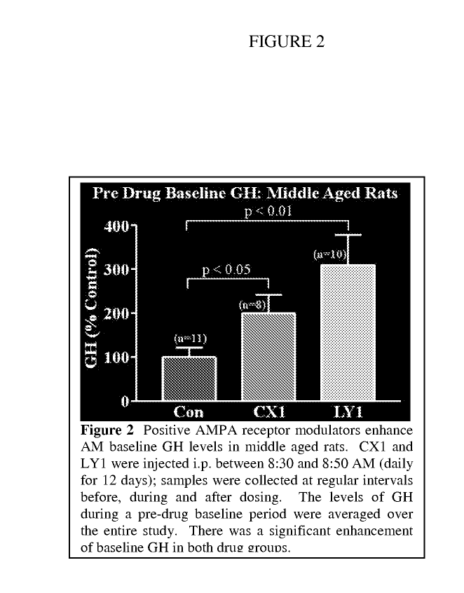

Study 2 In the second study, AMPA receptor modulators were tested in middle aged rats (see Methods). Twelve rats were divided equally among 3 treatment groups, those receiving: vehicle, CX1, and LY1 every day for 12 consecutive days. Samples were collected for ELISA analysis at regular intervals before, during and after drug administration. The timing of sample collection in this study was not optimal for defining an entire post-drug GH pulse, but it did pick up the peak and tail end of a major morning GH pulse that began prior to dosing. This is referred to as the pre-drug baseline GH. Paired comparisons of baseline GH between groups revealed a striking elevation of GH in each of the drug treatment groups relative to controls. FIG. 2 shows the average increases across the 12 day treatment period as a percentage of control. Relative to vehicle controls, baseline GH levels were increased by approximately 100% (p<0.05) and 200% (p<0.01) in CX1 and LY1-treated rats, respectively.

### C. Results—DHEA and Cortisol

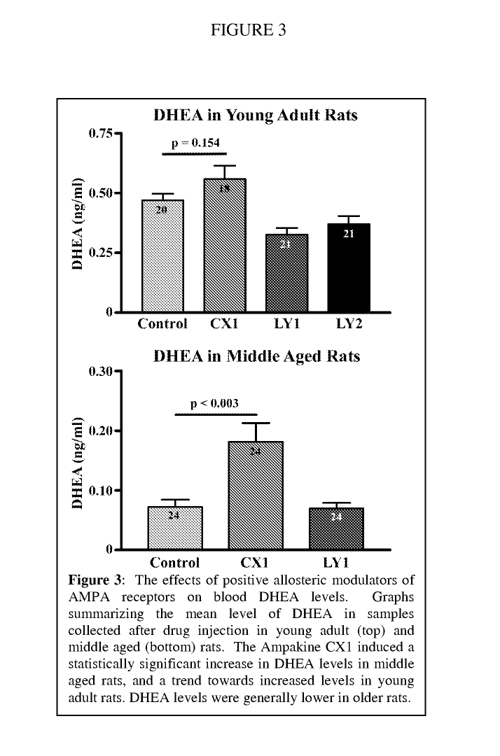

Administration of an Ampakine elevated levels of DHEA in rats. While there was a statistically non-significant trend towards increased DHEA in young rats, Ampakine treatment induced a large, significant increase in middle-aged rats (p<0.003, t-test). This effect is shown if FIG. 3. Treatment of rats with modulators from Eli Lilly did not have this effect, and in fact appeared to reduce DHEA levels in the young adult rats. This is in contrast to the changes we measured in GH levels, in which both types of positive AMPA modulators had similar effects. Another notable feature of these data is that overall levels of DHEA are reduced in the older, relative to younger, rats. This accords well with published data and suggests that the greater degree of enhancement achieved with the Ampakine in older animals is due in part to this falling baseline.

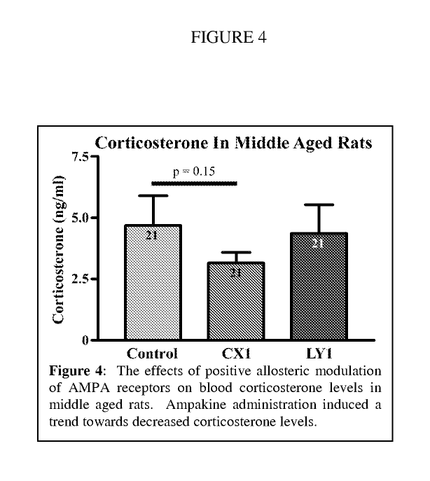

A trend towards decreased levels of corticosterone was observed in middle-aged rats treated with an Ampakine. FIG. 4 shows the average levels of corticosterone in samples collected after injection of positive modulators in Study 2. There appeared to be no difference between control animals and those treated with the Lilly compound. While not statistically significant at present (p=0.15), the trend towards decreased levels of corticosterone after Ampakine treatment is being analyzed further by deriving the cortisol-DHEA ratio per sample. This may reveal a significant change in the relationship of these two adrenal hormones after treatment with Ampakines.

### D. Discussion

The data we obtained demonstrate that in vivo administration of AMPA receptor positive modulators enhances GH secretion. This effect was obtained in both young and middle aged rats dosed once a day for 4 and 12 days, respectively. These results add to a variety of data implicating glutamatergic synapses in the control of endocrine function. Moreover, they suggest that facilitation of AMPA receptors is a viable means of normalizing endocrine functional deficits that stem from declines in glutamatergic function.

From the above results, it can be seen that glutamate regulates the secretion of cortisol, sex hormones, DHEA, and other hormones. In addition to acute effects of facilitating AMPA receptors, secondary benefits may be realized by upregulation of the neurotrophin BDNF, which is known to occur in several glutamatergic circuits after application of Ampakines and other classes of positive modulators. BDNF expression in hypothalamus may enhance the viability of glutamatergic neurons controlling hormone releasing factor release. In addition, it is known that BDNF enhances the viability of primary neurons in the hippocampus, which sits atop the hypothalamic pituitary axis in the control of many hormones, particularly stress hormones. Hippocampal neuron loss is, in part, causally linked with the dysregulation of stress hormone responses, therefore, beneficial effects on endocrine function may be obtained by upregulation of BDNF in hippocampus using Ampakines or other modulators.

Our results are particularly significant in the context of recent trends in hormone replacement therapy. Supplementation of older individuals with GH, testosterone, estrogen, thyroxine, DHEA and other hormones has been promoted as a means of offsetting age related declines in somatic and central nervous system function. While this approach has seen some success, concerns exist with most hormones that side effects may outweigh the benefits. The potential for side effects is in part due to the inability of supplementation to match the temporal and concentration characteristics of hormone pulses regulated by the hypothalamic-pituitary axis. Allosteric facilitation of AMPA receptors presents a strategy for endocrine enhancement that preserves the integrity of circadian linked pulses of hormones, both in their timing and in their amplitudes. This is important in terms of keeping hormone pulses within their physiological limits, and in terms of persevering temporal interactions between hormone systems that may be perturbed by direct replacement of a hormone. The rationale for using AMPA receptor modulators in this context is not unlike that supporting their use as cognitive enhancers in that they enhance endogenous activity rather than directly inducing excitation or inhibition. This strategy will realize many of the goals of hormone replacement with a much lower chance of side effects.

Our present data extend the known effects of Ampakines to include elevations in DHEA and decreases in cortisol. In total, the results show that positive modulation of AMPA receptors can be used to normalize the activity of a subset of hormones with critical functions in the ageing process. It has been suggested that GH, DHEA and cortisol represent the three most important hormones systems in terms of their role in normal ageing processes and the exacerbation of age-related disease states such as Alzheimer's disease. The normalization of this critical trio of hormones by a centrally active drug is thus highly significant. Moreover, given the interaction of GH, DHEA and cortisol with other hormone systems, and direct actions of Ampakines on hypothalamic cells governing their release, it is likely that Ampakines will normalize the levels of other hormones as well. This application demonstrates that facilitation of AMPA receptors can be used to normalize the activity of multiple hormone systems in a manner that is far superior to replacement therapy in terms of their circadian timing, amplitude, and interactions.

### BIBLIOGRAPHY

U.S. Pat. No. 6,620,808, and are hereby incorporated by reference.
U.S. Pat. No. 6,620,808, and are hereby incorporated by reference.
U.S. Pat. No. 6,620,808, and are hereby incorporated by reference.

It is evident from the above results and discussion that novel methods of conveniently regulating the endocrine system of a mammal are provided. Since the subject methods employ small, synthetic organic compounds, they do not suffer from the problems associated with delivery of larger peptide compounds, where, depending on the method of their production and physical characteristics, such compounds may be contaminated with various pathogens, require invasive administration, or suffer from other disadvantages. Furthermore, since the subject methods modulate the endocrine system through regulation of endogenous hormonal production, the possibility exists to achieve better regulation of the endocrine system than is obtainable with convention hormonal replacement therapy.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method for increasing the baseline circulatory levels of growth hormone in a mammalian host, the method comprising:
repeatedly administering to the host an ampakine, wherein the ampakine is a positive allosteric modulator of an AMPA receptor of the hypothalamus; whereby the baseline circulatory level of growth hormone in the mammalian host increases.

2. The method according to claim 1, where the mammalian host is a human.

3. The method according to claim 1, wherein said administering is by oral delivery.

4. The method according to claim 1, wherein said repeatedly administering occurs over a time period between one and twelve days.

5. The method according to claim 1, wherein said repeatedly administering occurs over a time period greater than twelve days.

6. A method for increasing the peak level of growth hormone in a mammalian host, the method comprising:
administering to the host an ampakine, wherein the ampakine is a positive allosteric modulator of an AMPA receptor of the hypothalamus; whereby the peak level of growth hormone in the mammalian host increases.

7. The method according to claim 6, where the mammalian host is a human.

8. The method according to claim 6, wherein said administering is by oral delivery.

9. A method for increasing the circulatory level of DHEA in a mammalian host, the method comprising: administering to the host an ampakine, wherein the ampakine is a positive allosteric modulator of an AMPA receptor of the hypothalamus; whereby the circulatory level of DHEA in the mammalian host increases.

10. The method according to claim 9, where the mammalian host is a human.

11. The method according to claim 9, wherein the mammalian host is suffering from a disease associated with an abnormally low circulatory level of DHEA.

12. The method according to claim 9, wherein the disease is associated with an age related decrease in DHEA levels.

13. The method according to claim 9, wherein said administering is by oral delivery.

14. A method for decreasing the circulatory level of cortisol in a mammalian host, the method comprising: administering to the host an ampakine, wherein the ampakine is a positive allosteric modulator of an AMPA receptor of the hypothalamus; whereby the circulatory level of cortisol in the mammalian host decreases.

15. The method according to claim 14, where the mammalian host is a human.

16. The method according to claim 14, wherein the mammalian host is suffering from a disease associated with an abnormally high circulatory level of cortisol.

17. The method according to claim 14, wherein the disease is associated with an age related increase in cortisol levels.

18. The method according to claim 14, wherein said administering is by oral delivery.
